(19) United States
(12) Patent Application Publication
(10) Pub. No.: US 2025/0229922 A1
(43) Pub. Date: Jul. 17, 2025

(54) METHODS, SYSTEMS, AND COMPUTER PROGRAM PRODUCT FOR GENERATING DISPENSE SETTINGS AND A CELL LAYOUT CONFIGURATION FOR A DRUG PRODUCT PACKAGING SYSTEM BASED ON DRUG PRODUCT CHARACTERISTICS

(71) Applicant: PARATA SYSTEMS, LLC, Durham, NC (US)

(72) Inventors: Charles Wayne Priest, III, Nokomis, FL (US); Vishnu Kodukulla, Apex, NC (US); Peter Anwar Saad, Holly Springs, NC (US)

(21) Appl. No.: 19/014,562

(22) Filed: Jan. 9, 2025

Related U.S. Application Data
(60) Provisional application No. 63/620,451, filed on Jan. 12, 2024.

(51) Int. Cl. B65B 5/10; G06F 16/2455

(52) U.S. Cl. CPC B65B 5/103; G06F 16/2455

(57) ABSTRACT

A method includes receiving information associated with a drug product; receiving an identifier for a drug product packaging system; querying a configuration database using the information and the identifier; and generating settings for packaging the drug product using the drug product packaging system responsive to querying the configuration database.

### CROSS-REFERENCE TO RELATED APPLICATION

This application claims priority to U.S. Provisional Patent Application No. 63/620,451, filed on Jan. 12, 2024, the disclosure of which is incorporated herein by reference in its entirety.

### BACKGROUND

The present disclosure relates generally to the identification of drug products and management of drug product packaging systems, and, in particular, to methods, systems, and computer program products for identifying drug products and creating a searchable database using the drug product information for use in managing drug product packaging systems.

Drug product packaging systems may be used in facilities, such as pharmacies, hospitals, long term care facilities, and the like to dispense medications to fill prescriptions. Configuring the settings to dispense a drug product in a drug product packaging system is typically a manual process in which the drug product is identified, information about the characteristics of the drug product is obtained from one or more information repositories and/or observing/measuring the drug product directly, and then manually adjusting the settings for dispensing the drug product in the drug product packaging system. Moreover, the configuration of where certain drug products are placed in the drug product packaging system is also generally a manual process in which an operator attempts to place the various drug products in cells in a way that improves the performance of the drug product packaging system. These drug product packaging systems may include systems designed to package medications in various container types including, but not limited to, pouches, vials, bottles, blistercards, and strip packaging. Strip packaging is a type of packaging wherein medications are packaged in individual pouches for administration on a specific date and, in some cases, at a specific time. Typically, individual pouches are removably joined together and often provided in rolls. The pouches can be separated from the roll when needed. Before a drug product is released to a customer, the contents of the drug product package may be validated to ensure that the customer is receiving the correct drug product.

### SUMMARY

In some embodiments of the inventive concept, a method comprises: receiving information associated with a drug product; receiving an identifier for a drug product packaging system; querying a configuration database using the information and the identifier; and generating settings for packaging the drug product using the drug product packaging system responsive to querying the configuration database.

In other embodiments, the information comprises an identity of the drug product.

In still other embodiments, querying the configuration database comprises querying the configuration database using the identity of the drug product and the identifier.

In still other embodiments, each of the plurality of characteristics comprises size, shape, color, imprint code, or scoring.

In still other embodiments, the imprint code comprises an indicium of medicinal strength, an indicium of an active ingredient, and an indicium of an inactive ingredient.

In still other embodiments, the shape comprises round, elliptical, capsule, triangular, oblong, oval, square, diamond, hexagon, octagon, pentagon, gelcap, non-uniform, and other.

In still other embodiments, the color comprises transparent and a plurality of colors.

In still other embodiments, the identity comprises a National Drug Code (NDC), a Drug Identification Number (DIN) identifier, or a Universal Product Code (UPC) identifier.

In still other embodiments, the settings comprise one or more of dispense channel dimensions for the drug product, a baffle arrangement for the drug product, or a dispense channel air pressure for the drug product.

In still other embodiments, the method further comprises: communicating the settings to the drug product packaging system; and initiating application of the settings on the drug product packaging system responsive to communicating the settings to the drug product packaging system.

In some embodiments of the inventive concept, a method comprises: receiving a dispensing history of a dispensary that identifies a plurality of drug products; receiving an identifier for a drug product packaging system; and generating a layout configuration that identifies placement of the plurality of drug products in cell locations in the drug product packaging system based on the dispensing history of the dispensary and the identifier for the drug product packaging system

In further embodiments, the method further comprises: querying a configuration database using an identity of one of the plurality of drug products to obtain size information for the one of the plurality of drug products; and determining a cannister size for the one of the plurality of drug products based on the size information; wherein generating the layout configuration comprises: generating the layout configuration based on the dispensing history and the cannister size for the one of the plurality of drug products.

In still further embodiments, determining the cannister size comprises: determining the cannister size for the one of the plurality of drug products based on the size information and package quantity information for the one of the plurality of drug products.

In still further embodiments, generating the layout configuration comprises: generating the layout configuration with ones of the plurality of drug products having higher dispense rates at ones of the cell locations that operate as fast or faster than other ones of the cell locations.

In still further embodiments, generating the layout configuration comprises: generating the layout configuration such that ones of the cell locations that contain a same one of the plurality of drug products are adjacent to one another.

In still further embodiments, generating the layout configuration such that the ones of the cell locations that contain the same one of the plurality of drug products are adjacent to one another comprises: generating the layout configuration such that the ones of the cell locations that contain the same one of the plurality of drug products are in a common drawer.

In still further embodiments, the identity comprises a National Drug Code (NDC), a Drug Identification Number (DIN), or a Universal Product Code (UPC) identifier.

In some embodiments of the inventive concept, a system comprises: a processor; and a memory coupled to the processor and comprising computer readable program code embodied in the memory that is executable by the processor to perform operations comprising: receiving information associated with a drug product; receiving an identifier for a drug product packaging system; querying a configuration database using the information and the identifier; and generating settings for packaging the drug product using the drug product packaging system responsive to querying the configuration database.

In some embodiments of the inventive concept, a system comprises: a processor; and a memory coupled to the processor and comprising computer readable program code embodied in the memory that is executable by the processor to perform operations comprising: receiving a dispensing history of a dispensary that identifies a plurality of drug products; receiving an identifier for a drug product packaging system; and generating a layout configuration that identifies placement of the plurality of drug products in cell locations in the drug product packaging system based on the dispensing history of the dispensary and the identifier for the drug product packaging system.

In some embodiments of the inventive concept, computer program product comprises: a non-transitory computer readable storage medium comprising computer readable program code embodied in the medium that is executable by a processor to perform operations comprising: receiving information associated with a drug product; receiving an identifier for a drug product packaging system; querying a configuration database using the information and the identifier; and generating settings for packaging the drug product using the drug product packaging system responsive to querying the configuration database.

In some embodiments of the inventive concept, a computer program product comprises: a non-transitory computer readable storage medium comprising computer readable program code embodied in the medium that is executable by a processor to perform operations comprising: receiving a dispensing history of a dispensary that identifies a plurality of drug products; receiving an identifier for a drug product packaging system; and generating a layout configuration that identifies placement of the plurality of drug products in cell locations in the drug product packaging system based on the dispensing history of the dispensary and the identifier for the drug product packaging system.

Other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including an Artificial Intelligence (AI) assisted drug product analysis and packaging system configuration system in accordance with some embodiments of the inventive concept;

FIG. 2 is a block diagram of the AI assisted drug product analysis system of FIG. 1 in accordance with some embodiments of the inventive concept;

FIG. 3 is a block diagram of a convolutional neural network for determining or confirming an identity of a drug product in accordance with some embodiments of the inventive concept;

FIG. 4 is a block diagram of a skip connection arrangement between convolutional layers of the convolutional neural network of FIG. 3 in accordance with some embodiments of the inventive concept;

FIG. 5 is a block diagram that illustrates drug product image pre-processing in accordance with some embodiments of the inventive concept;

FIG. 6 is a block diagram that illustrates pre-filtering or pre-categorization of drug product information based on characteristics or features in accordance with some embodiments of the inventive concept;

FIG. 7 is a flowchart that illustrates operations for performing common drug database management in accordance with some embodiments of the inventive concept;

FIG. 8 is a block diagram of a common drug database including heterogeneously obtained drug product information in accordance with some embodiments of the inventive concept;

FIG. 9 is a flow chart that illustrates operations for generating dispense settings for a drug product packaging system based on drug product characteristics in accordance with some embodiments of the inventive concept;

FIG. 10 is a flowchart that illustrates operations for generating a cell layout configuration for a drug product packaging system based on drug product characteristics in accordance with some embodiments of the inventive concept;

FIG. 11 is a data processing system that may be used to implement one or more servers in the AI assisted drug product analysis and packaging system configuration system of FIG. 1 in accordance with some embodiments of the inventive concept; and

FIG. 12 is a block diagram that illustrates a software/hardware architecture for use in the AI assisted drug product analysis and packaging system configuration system of FIG. 1 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “data processing facility” includes, but it is not limited to, a hardware element, firmware component, and/or software component. A data processing system may be configured with one or more data processing facilities.

The term “drug product packaging system,” as used herein, refers to any type of pharmaceutical dispensing system including, but not limited to, automated systems that fill vials, bottles, containers, pouches, blistercards, or the like with drug product, semi-automated systems that fill vials, bottles, containers, pouches, blistercards, or the like with drug product, and any combination of automated and semi-automated systems for filling a drug product package with drug product. Drug product packaging system also includes packaging systems for pharmaceutical alternatives, such as nutraceuticals and/or bioceuticals.

The terms “pharmaceutical” and “medication,” as used herein, are interchangeable and refer to medicaments prescribed to patients either human or animal. A pharmaceutical or medication may be embodied in a variety of ways including, but not limited to, pill form capsule form, tablet form, and the like.

The term “drug product” refers to any type of medicament that can be packaged within a vial, bottle, container, pouch, blistercard, or the like by automated and semi-automated drug product packaging systems including, but not limited to, pills, capsules, tablets, caplets, gel caps, lozenges, and the like. Drug product also refers to pharmaceutical alternatives, such as nutraceuticals and/or bioceuticals. Example drug product packaging systems including management techniques for fulfilling packaging orders are described in U.S. Pat. No. 10,492,987, the disclosure of which is hereby incorporated herein by reference.

The term “drug product package” refers to any type of object that can hold a drug product including, but not limited to, a vial, bottle, container, pouch, blistercard, or the like.

Embodiments of the inventive concept are described herein in the context of a drug product analysis and package validation engine that includes one or more Artificial Intelligence (AI) models. It will be understood that embodiments of the inventive concept are not limited to particular implementations of the drug product analysis engine and various types of AI systems may be used including, but not limited to, a multi-layer neural network, a deep learning system, a natural language processing system, and/or computer vision system Moreover, it will be understood that the multi-layer neural network is a multi-layer artificial neural network comprising artificial neurons or nodes and does not include a biological neural network comprising real biological neurons. Embodiments of the inventive concept may be implemented using multiple AI systems or may be implemented by combining various functionalities into fewer or a single AI system. The AI models described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the AI training process to improve the efficiency of a computer system when the computer system operates in an inference mode to make an inference, prediction, classification, suggestion, or the like in response to input information or data provided thereto.

When drug products are packaged for delivery to a customer, drug product packaging systems are used that are designed to package the drug products into various container types including, but not limited to, pouches, vials, bottles, blistercards, and strip packaging. Configuring the dispense settings on these drug product packaging systems is generally based on the characteristics of the drug products being dispensed. Information on the different drug products may be located in a variety of different information repositories including public repositories and privately maintained repositories by pharmacies, hospitals, and the like. These information repositories, however, generally lack details on the size and geometry of the drug products, which is used in determining the settings for the drug product packaging systems. It may be both processor and labor intensive to collect and gather the size and geometry information for large numbers of different drug products to compile a sufficiently comprehensive drug product characteristic information database that can be efficiently used to manage drug product packaging systems, e.g., configure dispense settings thereon.

When drug products are packaged for delivery to a customer using a drug product packaging system, a validation or auditing process may be performed to ensure that the drug product that was packaged corresponds to the prescription for the patient. In some instances, drug product packaging entities may use an AI system to analyze drug product images and/or other drug product information to predict the corresponding drug product identifier, such as a National Drug Code (NDC), Drug Identification Number (DIN), and/or Universal Product Code (UPC), or to confirm whether the appearance of the drug product matches the appearance of the expected drug product determined by the drug identifier of the drug product included in the prescription. In some embodiments, other identifiers may be used for drug products, such as vitamins, nutraceuticals, veterinary drugs, and the like.

Some embodiments of the inventive concept stem from a realization that a data structure, such as a database containing drug product identifiers along with their associated characteristics, and/or drug product packaging system dispensing setting information for each of the drug products, may be configured in a computer readable storage medium where it can be accessed by query. This data structure or database can be populated with information from drug product information repositories, e.g., commercially available and/or proprietary databases, and supplemented with drug product size and/or geometry information obtained, for example, when validating drug products during the packaging process. Advantageously, one or more of the information items obtained from the drug product information repository and/or the drug product size and/or geometry information obtained from validating drug products in the packaging process may be selected to have different quantization levels, e.g., 64-bit, 32-bit, etc. from other data items to reduce the size of the data structure that is stored in the computer-readable memory. For example, the quantization levels for representing drug products may be varied depending on a degree of precision desired for distinguishing between various ones of these entities in the same category, i.e., distinguishing one medication from another or distinguishing between like entities, e.g., labeled and generic versions of the same medication.

Thus, the data structure or database can be effectively populated through a drug product information crowd sourcing approach where drug product characteristics can be collected across numerous drug product packaging systems to supplement the information collected from drug product information repositories. As the information in the data structure or database may be accessed, in some embodiments, using a query, the processing time and memory resources may be reduced relative to performing searches across multiple information repositories seeking characteristic information on drug products, such as size, geometry, etc., that may be difficult to find. The data structure or database may be used as a resource for managing or configuring the settings for dispensing the various drug products to improve the operating efficiency in dispensing drug product.

In addition to configuring the settings on a drug product packaging system, drug products may be placed into a drug product packaging system in particular locations. Some embodiments of the inventive concept may provide a system that generates a layout configuration for a drug product packaging system that identifies locations for the various drug products that will improve the efficiency of dispensing the drug products and maintaining the drug products in the packaging system, e.g., managing re-fills of the drug products. According to some embodiments, the drug layout configuration, i.e., the placement of drug products in cell locations, is based on a dispensing history for the drug products, which provides information on, for example, a pharmacy formulary, dispensing frequency, and/or workflow.

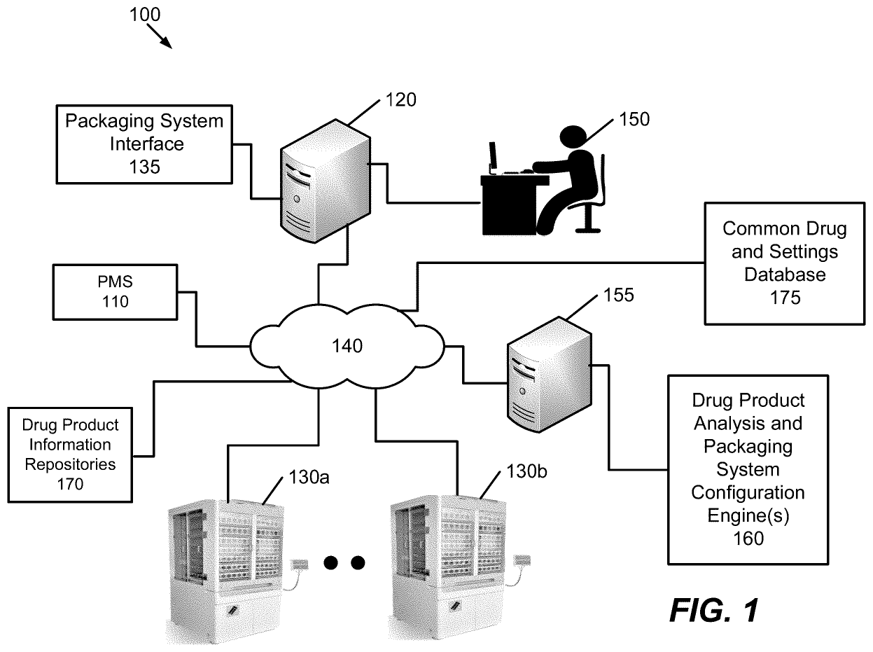

Referring to FIG. 1, a communication network 100 including an AI assisted drug product analysis and packaging system configuration system, in accordance with some embodiments of the inventive concept, comprises a pharmacy management system (PMS) or host system 110, a packaging system server 120, a drug product analysis and packaging system configuration server 155, and one or more drug product packaging systems 130a and 130b that are coupled via a network 140 as shown.

The PMS system 110 may be configured to manage and fill prescriptions for customers. As used herein, PMS systems may be used in pharmacies or may be used generally as batch-generating systems for other applications, such as dispensing nutraceuticals or bioceuticals. The PMS system 110 may be associated with a variety of types of facilities, such as pharmacies, hospitals, long term care facilities, and the like. The PMS system or host system 110 may be any system capable of sending a valid prescription to the one or more drug product packaging systems 130a and 130b. The packaging system server 120 may include a packaging system interface module 135 and may be configured to manage the operation of the drug product packaging systems 130a and 130b. For example, the packaging system server 120 may be configured to receive packaging orders from the PMS system 110 and to identify which of the drug product packaging systems 130a and 130b should be used to package particular individual orders or batches of orders. In addition, the packaging system server 120 may be configured to manage the operations of the drug product packaging systems 130a and 130b. For example, the packaging system server 120 may be configured to manage the inventory of drug product available through each of the drug product packaging systems 130a and 130b, to manage the drug product dispensing canisters assigned or registered to one or more of the drug product packaging systems 130a and 130b, to manage the operational status generally of the drug product packaging systems 130a and 130b, and/or to manage reports regarding the status (e.g., assignment, completion, etc.) of packaging orders, drug product inventory, order billing, and the like. A user 150, such as a pharmacist or pharmacy technician, may communicate with the packaging system server 120 using any suitable computing device via a wired and/or wireless connection. Although the user 150 is shown communicating with the packaging system server 120 via a direct connection in FIG. 1, it will be understood that the user 150 may communicate with the packaging system server 120 via one or more network connections (e.g., via the network 140). The user 150 may interact with the packaging system server 120 to approve or override various recommendations made by the packaging system server 120 in operating the drug product packaging systems 130a and 130b. The user 150 may also initiate the running of various reports as described above for the drug product packaging systems 130a and 130b. Although only two drug product packaging systems 130a and 130b are shown in FIG. 1, it will be understood that more than two drug product packaging systems may be managed by the packaging system server 120.

The AI assisted drug product analysis and packaging system configuration system may include the drug product analysis and packaging system configuration server 155, which includes a drug product analysis and packaging system configuration module 160 configured to, for example, facilitate validation of a packaged and/or unpackaged drug product, collect drug product identifiers and associated drug product characteristics from the validation process, compile the collected information along with drug product identities and characteristics obtained from drug product information repositories 170 into a common drug and settings database 175, and generate drug product dispense settings and/or drug product layout configurations for one or more drug product packaging systems 130a and 130b. In some embodiments of the inventive concept, the drug product information repositories may include, but are not limited to, commercially available databases and/or proprietary databases. In some embodiments, the drug product analysis and packaging system configuration server 155 and drug product analysis and packaging system configuration module 160 may represent a single AI system that is trained for validating a packaged or unpackaged drug product when operating in inference mode. In other embodiments, the drug product analysis and packaging system configuration server 155 and drug product analysis and packaging system configuration module 160 may represent one or more AI systems that are trained and operated in inference mode by pre-filtering or pre-categorizing drug products based on one or more characteristics thereof, which may allow the drug products to be sorted into categories or groups. The drug product analysis and packaging system configuration server 155 may, therefore, represent the one or more AI engines or models that respectively correspond to the drug product categories or groups that are identified through the pre-filtering or pre-categorization. Smaller AI engines or models that correspond to the various categories or groups of drug products may be more efficient to train as new drug products are developed and added to the system and may be more relevant to individual drug product distribution entities that may only distribute a subset of drug products to their customers. The one or more AI engines or models may also be configured to generate modified images of drug product packages to account for differences in packaging systems. Such AI systems are described, for example, in PCT International Publication Nos. WO 2022/177954 and WO 2022/165135 the disclosures of which are hereby incorporated herein by reference.

It will be understood that the division of functionality described herein between the packaging system server 120/packaging system interface module 135 and the drug product analysis and packaging system configuration server 155/drug product analysis and packaging system configuration module 160 is an example. Various functionality and capabilities can be moved between the packaging system server 120/packaging system interface module 135 and the drug product analysis and packaging system configuration server 155/drug product analysis and packaging system configuration module 160 in accordance with different embodiments of the inventive concept. Moreover, in some embodiments, the packaging system server 120/packaging system interface module 135 and the drug product analysis and packaging system configuration server 155/drug product analysis and packaging system configuration module 160 may be merged as a single logical and/or physical entity.

A network 140 couples the drug product packaging systems 130a and 130b, the PMS system 110, the packaging system server 120, the drug product analysis and packaging system configuration server 155, the common drug and settings database 175, and the drug product information repositories 170 to one another. The network 140 may be a global network, such as the Internet or other publicly accessible network. Various elements of the network 140 may be interconnected by a wide area network, a local area network, an Intranet, and/or other private network, which may not be accessible by the general public. Thus, the network 140 may be a communication network and/or may represent a combination of public and private networks or a virtual private network (VPN). The network 140 may be a wireless network, a wireline network, or may be a combination of both wireless and wireline network.

The AI assisted drug product analysis service and packaging system setting and configuration service provided through the drug product analysis and packaging system configuration server 155, and drug product analysis and packaging system configuration module 160, in some embodiments, may be implemented as a cloud service. In some embodiments, the AI assisted drug product analysis service and packaging system setting and configuration service may be implemented as a Representational State Transfer Web Service (RESTful Web service).

Although FIG. 1 illustrates an example communication network that includes AI assisted drug product analysis service and packaging system setting and configuration systems, it will be understood that embodiments of the inventive subject matter are not limited to such configurations, but are intended to encompass any configuration capable of carrying out the operations described herein.

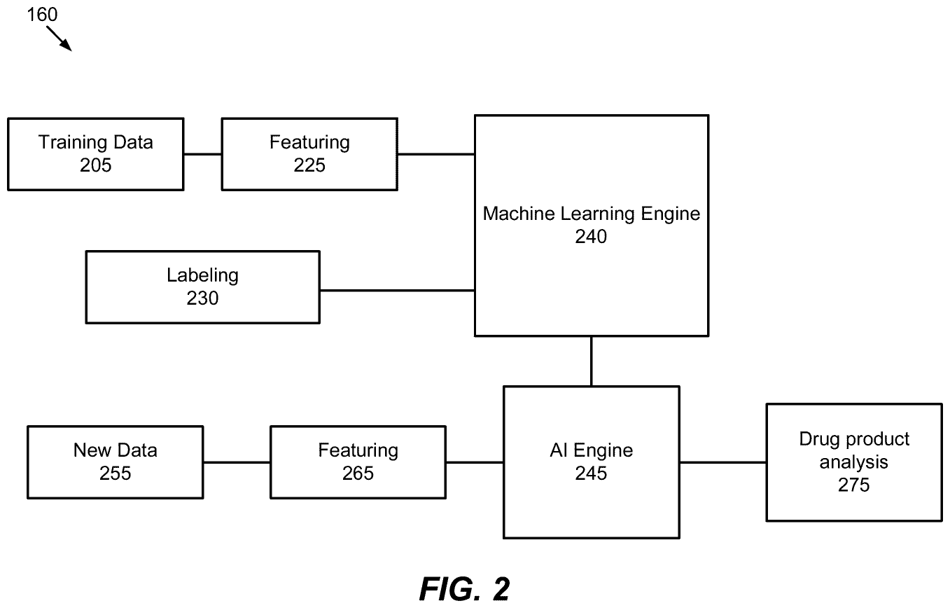

As described above, the drug product analysis and packaging system configuration server 155 and drug product analysis and packaging system configuration module 160 may represent one or more AI systems and/or control logic that may, for example, facilitate validation of packaged and/or unpackaged drug products, may configure and manage a data structure in which commercially available drug product information may be supplemented with characteristics of the drug product, e.g., size and geometry information, which may be obtained from package validation process performed at one or more drug product packaging systems, and/or generate a layout configuration for a drug product packaging system based, for example, on a drug product dispensing history. FIG. 2 is a block diagram of the drug product analysis engine(s) module 160 embodied as an AI system, including one or more AI engines or models, such as machine learning systems, which can be used to detect packaged and/or unpackaged drug products, and/or to identify these drug products that have been detected by an identifier, such as NDC, DIN, and/or UPC, and/or confirm or verify whether a drug product matches an expected or target drug product. The AI system of FIG. 2 may be representative of a single AI engine or model that may be used for identifying drug products that correspond to a single group based on pre-filtering or pre-categorization of the drug products based on one or more characteristics thereof. Thus, the architecture of the AI system of FIG. 2 may be duplicated to form separate AI systems to detect packaged and/or unpackaged drug products, and to identify these drug products that have been detected by an identifier, such as NDC, DIN, and/or UPC respectively. As shown in FIG. 2, the drug product analysis and packaging system configuration module 160 may include both training modules and modules used for processing new data on which to detect and/or identify packaged and/or unpackaged drug products in an image. The modules used in the training portion of the drug product analysis engine(s) module 160 may include a training data module 205, a featuring module 225, a labeling module 230, and a machine learning engine 240.

The training data module 205 may be configured to obtain and/or store training data, which may comprise one or more images of packaged and/or unpackaged drug products along with additional information or data associated with each of the drug products, such as characteristics of the drug product. The training data stored by the training data module 205 may also include an identifier, such as a NDC, DIN, and/or UPC for each of the drug products. While a machine learning architecture is shown in FIG. 2, other embodiments may use in an artificial neural network in addition to or in place of the machine learning system embodiment of FIG. 2. The featuring module 225 is configured to identify the individual independent variables that are used by the drug product analysis engine(s) module 160 to detect and/or identify one or more drug products in, for example, an image of a packaged and/or unpackaged drug product, which may be considered dependent variable(s). The training data may be generally unprocessed or formatted and include extra information in addition to drug product and/or drug product packaging information. For example, the training data may include account codes, business address information, and the like, which can be filtered out by the featuring module 225. The features extracted from the training data may be called attributes and the number of features may be called the dimension. According to some embodiments of the inventive concept, one or more characteristics of a drug product may be used in pre-filtering or pre-categorization of the drug product as will be described below. Those characteristics need not be feature candidates identified by the featuring module 225 as the drug products for which this AI engine or model is being trained are known to have those one or more characteristics.

The labeling module 230 may be configured to assign defined labels to the training data and to the detected and/or identified drug products to ensure a consistent naming convention for both the input features and the generated outputs. The machine learning engine 240 may process both the featured training data, including the labels provided by the labeling module 230, and may be configured to test numerous functions to establish a quantitative relationship between the featured and labeled input data and the generated outputs. The machine learning engine 240 may use modeling techniques to evaluate the effects of various input data features on the generated outputs. These effects may then be used to tune and refine the quantitative relationship between the featured and labeled input data and the generated outputs. The tuned and refined quantitative relationship between the featured and labeled input data generated by the machine learning engine 240 is output for use in the AI engine 245. The machine learning engine 240 may be referred to as a machine learning algorithm.

The modules used to detect packaged and/or unpackaged drug products, to identify these drug products that have been detected by an identifier, such as NDC, DIN, and/or UPC in the image, and/or to verify that these drug product(s) match one or more target drug product(s) include the new data module 255, the featuring module 265, the AI engine 245, and the drug product analysis module 275. The new data 255 may be the same data/information as the training data 205 in content and form except the new data 255 will be used for an analysis of a new packaged and/or unpackaged drug product rather than for training purposes. Likewise, the featuring module 265 performs the same functionality on the new data 255 as the featuring module 225 performs on the training data 205. The AI engine 245 may, in effect, be generated by the machine learning engine 240 in the form of the quantitative relationship determined between the featured and labeled input data and the output drug product package content analysis. The AI engine 245 may, in some embodiments, be referred to as an AI model.

The AI engine 245 may be configured to identify one or more drug products based on their identifiers, such as their NDCs, DINs, and/or UPCs, and/or to verify that a drug product(s) matches a target drug product(s). The AI engine 245 may use a variety of modeling techniques to detect packaged and/or unpackaged drug products, and to identify these drug products that have been detected in the image by an identifier, such as NDC, DIN, and/or UPC, in accordance with different embodiments of the inventive concept including, but not limited to, a regression technique, a neural network technique, an Autoregressive Integrated Moving Average (ARIMA) technique, a deep learning technique, a linear discriminant analysis technique, a decision tree technique, a naïve Bayes technique, a K-nearest neighbors technique, a learning vector quantization technique, a support vector machine technique, and/or a bagging/random forest technique.

The drug product analysis module 275 may be configured to output the drug product identifier, such as NDC code, DIN code, and/or UPC code, for a packaged and/or unpackaged drug product image with the one or more drug products identified by way of one or more indicia, such as boundary boxes, along with the identifiers, such as NDCs, DINs, and/or UPCs, for the one or more drug products to a drug product package validation system. The drug product analysis module 275 may also output the drug product characteristics for each of the identified drug products, which may be stored in the common drug and settings database 175. In some embodiments, the drug product characteristics may include, but are not limited to, size, shape, color, imprint code, or scoring. The imprint code may comprise an indicium of medical strength, an indicium of an active ingredient, and/or an indicium of an inactive ingredient. The shape may comprise round, elliptical, and/or other. The color may comprise transparent and/or a plurality of colors.

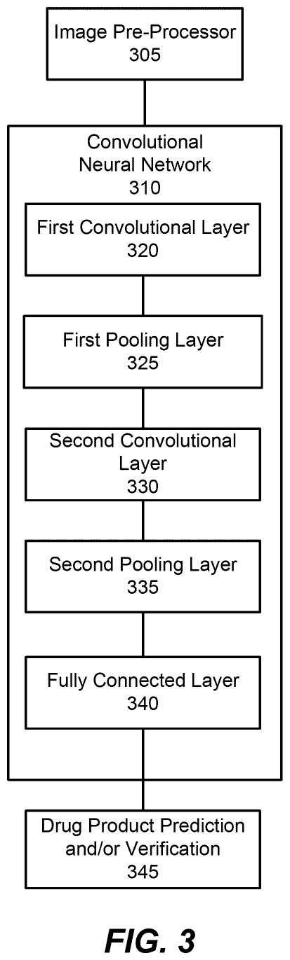

As described above, the drug product analysis and packaging system configuration server 155 and the drug product analysis and packaging system configuration module 160 may represent one or more AI systems that may, for example, facilitate validation of a packaged and/or unpackaged drug product, collect drug product identifiers and associated drug product characteristics from the validation process, compile the collected information along with drug product identities and characteristics obtained from drug product information repositories 170 into a common drug and settings database 175, and generate drug product dispense settings for one or more drug product packaging system. FIG. 3 is a block diagram of the drug product analysis and packaging system configuration module 160 for implementing an AI system, by way of a neural network, that can be used to supplement and/or replace the machine learning embodiments of FIG. 2 to detect packaged and/or unpackaged drug products, to identify these drug products that have been detected in an image by an identifier, such as NDC, DIN, and/or UPC, and/or to verify that a drug product(s) matches a target drug product(s). In the example embodiment of FIG. 3, the neural network is a convolutional neural network. It will be understood, however, that the AI system for detecting packaged and/or unpackaged drug products, to identify these drug products that have been detected by an identifier, such as NDC, DIN, and/or UPC, and/or to verify that a drug product(s) matches a target drug product(s) may also be embodied as a fully connected neural network in accordance with other embodiments of the inventive concept. A convolutional neural network may, however, be useful when processing or classifying images due to the large number of pixels and the resulting large number of weights to manage in the neural network layers. A convolutional neural network may reduce the main image matrix to a matrix having a lower dimension in the several layers including hidden layers and activation layers through convolution, which reduces the number of weights used and reduces the impact on training time. The final layer may use a softmax function as the activation function in the output layer, which may predict a multinomial probability distribution that may match identifiers, such as NDC, DIN, and/or UPC labels.

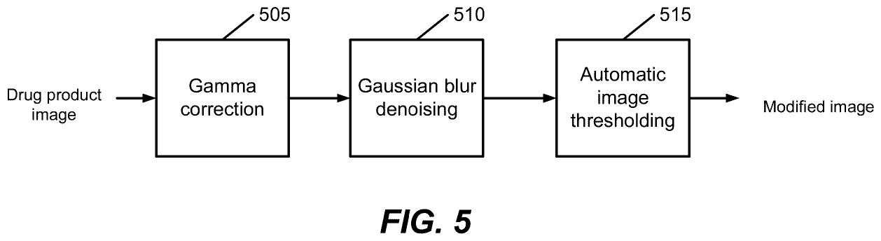

Referring now to FIG. 3, an image pre-processor 305 may receive one or more images of a packaged and/or unpackaged drug product. As will be described below with reference to FIG. 5, the image pre-processor may perform various corrections to the image data including, for example, gamma correction, noise reduction, and/or image segmentation. The pre-processed drug product image, which may be an image represented by a matrix of dimension A×B×3, where the number 3 represents the colors red, green, and blue, may then be provided to the convolutional neural network 310. As shown in FIG. 3, the convolutional neural network 310 includes first and second convolutional layers 320 and 330 along with first and second pooling layers 325 and 335. Each of the convolutional layers 320 and 330 is a matrix of a dimension smaller than the input matrix and may be configured to perform a convolution operation with a portion of the input matrix having the same dimension. The sum of the products of the corresponding elements is the output of the convolutional layer. The output of each of the convolutional layers may also be processed through a rectified linear unit operation in which any number below 0 is converted to 0 and any positive number is left unchanged. The convolutional neural network 310 further includes first and second pooling layers 325 and 335. The pooling layers 325 and 335 may each be configured to filter the output of the convolutional layers 320 and 330, respectively, by performing a down sampling operation. The size of the pooling operation or filter is smaller than the size of the input feature map. In some embodiments, it is 2×2 pixels applied with a stride of 2 pixels. This means that the pooling layer will always reduce the size of each feature map by a factor of 2, e.g., each dimension is halved, reducing the number of pixels or values in each feature map to one quarter the size. For example, a pooling layer applied to a feature map of 6×6 (36 pixels) will result in an output pooled feature map of 3×3 (9 pixels). The final output layer is a normal fully-connected neural network layer 340, which gives the output as a predicted drug product identifier, such as NDC, DIN, and/or UPC or drug product verification 345.

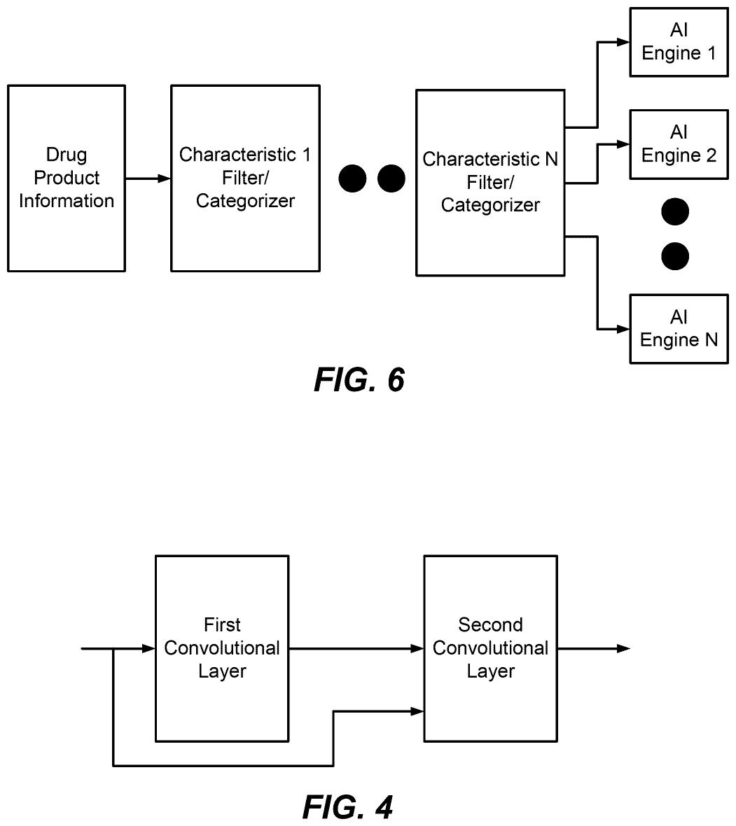

In some embodiments of the inventive concept, the convolutional neural network 310 may be a residual neural network in which skip connections are used between the convolutional layers 320 and 330. An example of the skip connection is shown in FIG. 4. Specifically, in a skip connection a convolutional neural network involves a convolutional layer receiving as an input both the output of a previous convolutional layer and the input to the previous convolutional layer.

It will be understood that while two convolutional layers 320 and 330 are shown in in the example convolutional neural network 310 of FIG. 3 for purposes of illustration, a convolutional neural network according to various embodiments of the inventive concept may contain numerous convolutional layers and may exceed 100 layers in some embodiments.

In some embodiments, the features of the training data used during training of the machine learning engine 240 may be evaluated to determine their relative impact on the accuracy in generating the drug product identifier, such as NDC code, DIN code, and/or UPC code, for a packaged and/or unpackaged drug product image with the one or more drug products identified by way of one or more indicia, such as boundary boxes, along with the identifiers, such as NDCs, DINs, and/or UPCs, for the one or more drug products, which are provided to the drug product analysis module 275. One or more features of the training data having a lesser impact on the accuracy of the output to the drug product analysis module 275 than other features may be removed from the training data to reduce the overall dimensionality of the training data set used to train the machine-learning engine 240. By sparsifying the features used during training and/or during inference mode of the machine learning engine 240 and/or the AI engine 245, the AI model may execute on one or more processors with decreased memory and computational requirements (e.g., processor speed and availability).

In some embodiments in a neural network is used to generate a predicted drug product identifier, such as NDC, DIN, and/or UPC or drug product prediction and/or verification 345, one or more nodes or paths of the neural network may be removed through setting certain parameters or weights to zero based on the sparsification of the features used during training and during inference mode. As a result, the sparsified neural network may run more efficiently and use fewer memory resources.

In some embodiments, one or more of the features used in training or during inference mode of machine learning engine 240 may be selected to have different quantization levels, e.g., 64-bit, 32-bit, etc. to reduce the size and complexity of the machine learning engine 240 and the AI engine 245. The quantization level may be adjusted to achieve a desired inference accuracy rate.

As described above, the drug product image may undergo pre-processing to perform various corrections to the image data. Referring now to FIG. 5, a gamma correction module 505 may perform gamma correction on the drug product image to generate a gamma corrected image. One or more cameras may make an image darker; the gamma correction may brighten the image to allow the convolutional neural network 310 to better recognize the edges of various elements displayed in the image. Gamma correction may be embodied as a power law transform, except for low luminosities where it may be linear to avoid having an infinite derivative at luminance zero. This is the traditional nonlinearity applied for encoding SDR images. The exponent or “gamma,” may have a value of 0.45, but the linear portion of the lower part of the curve may make the final gamma correction function to be closer to a power low exponent of 0.5, i.e., a square root transform; therefore, the gamma correction may comply with the DeVries-Rose law of brightness perception. The gaussian blur denoising module 510 is used to perform gaussian blur denoising on the gamma corrected image to generate a reduced noise image. The gaussian blur denoising module or filter 510 may be a linear filter. It may be used to blur the image and/or to reduce the noise. Two gaussian blur denoising filters 510 may be used such that the outputs are subtracted for “unsharp masking” (edge detection). The gaussian blur denoising module or filter 510 may blur edges and reduce contrast. The Median filter is a non-linear filter that may be used as a way to reduce noise in an image. The automatic image thresholding module 515 may perform automatic image thresholding on the reduced noise image to generate a foreground-background separated image. Thresholding is a technique used in image segmentation applications. Thresholding involves the selection of a desired gray-level threshold value for separating objects of interest in an image from the background based on their gray-level distribution. The Otsu method is a type of global thresholding that depends only on the gray value of the image. The Otsu method is a global thresholding selection method, which involves computing a gray level histogram. When applied in only one dimension an image may not be sufficiently segmented. A two-dimensional Otsu method may be used that is based on both the gray-level threshold of each pixel as well as its spatial correlation information with the neighborhood surrounding the pixel. As a result, the Otsu method may provide satisfactory segmentation when applied to noisy images. The output image from the pre-processing modules of FIG. 5 may be applied to a drug product analysis and validation engine, such as the convolutional neural network 310 of FIG. 3.

FIG. 6 is a block diagram that illustrates pre-filtering or pre-categorization of drug product information based on characteristics or features in accordance with some embodiments of the inventive concept. The pre-filtering or pre-categorization of the drug products based on one or more of their characteristics may allow the drug products to be sorted or divided into groups. As shown in FIG. 6, the drug product information may be processed using one or more filters or categorizers. In the example of FIG. 6, N filters or categorizers are shown. The number of filters or categorizers may be based on the number of characteristics associated with the drug products. In some embodiments, the drug product characteristics may comprise size, shape, color, imprint code, and/or scoring. The imprint code characteristic may comprise an indicia or medicinal strength, an indicium of an active ingredient, and/or an indicium of an inactive ingredient. The shape characteristic may be round, elliptical, and/or other. The color characteristic may be any of a plurality of colors and/or transparent. Multiple AI engines or models, such as those described above with respect to FIGS. 2 and 3, may be developed that respectively correspond to the different drug product groups that are generated based on the pre-categorization or pre-filtering. In the example shown in FIG. 6, N AI engines are shown. If two characteristics are used with each being able to assume two unique values, then four AI engines may be used corresponding to all four value combinations of the two drug product characteristics used in the pre-categorization or pre-filtering process. To predict an identifier, such as an NDC, DIN, and/or UPC for a drug product and/or to verify whether a drug product matches a target drug product, the AI engine or model that corresponds to the group that the drug product falls into based on the pre-categorization or pre-filtering may be selected and the drug product information for that drug product is provided to the selected AI engine or model. The AI engine or model may then predict the identifier, such as an NDC, DIN, and/or UPC code and/or verify whether a drug product matches a target drug product based on the information associated with the drug product in view of its training. Many drug product distribution entities may only distribute a small subset of the total number of drug products covered by all N AI engines or models shown in FIG. 6. Thus, according to some embodiments of the inventive concept, a drug product distribution entity may only be granted access to those AI engines or models that are associated with the groupings or categories of drug products that encompass the drug products that the drug product distribution entity distributes. The access may be, for example, via a cloud service and/or the appropriate AI engines or models may be provided to the drug product distribution entity for execution on the drug product distribution entity's own platform.

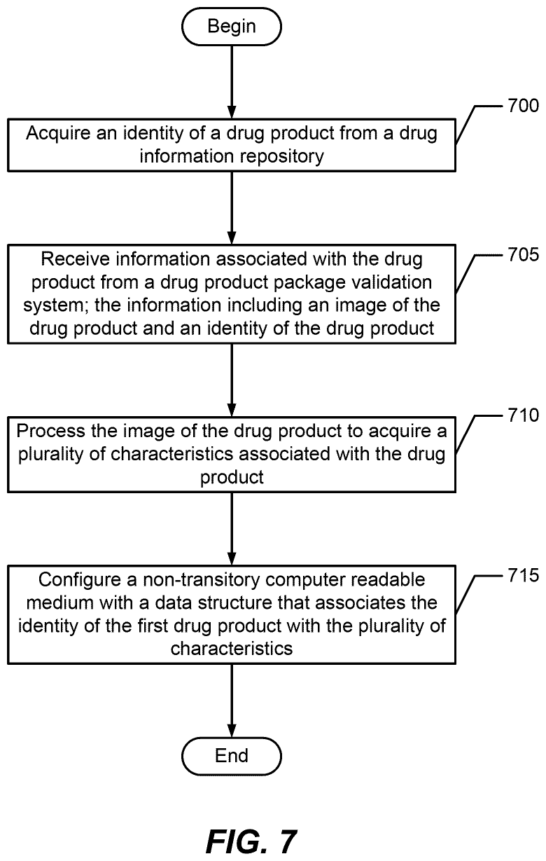

FIG. 7 is a flowchart that illustrates operations for performing common drug database management in accordance with some embodiments of the inventive concept. Referring now to FIG. 7, operations begin at block 700 where an identity of a drug product from a drug information repository is received. As described above, the drug information repository may be one or more commercially available and/or proprietary databases. Thus, the common drug and setting database 175 may include records for one or more drug products that are obtained from these commercially available and/or proprietary databases. But as further described above, the information available from these databases may not include characteristics for the drug products, such as size, shape, color, and the like. Some embodiments of the inventive concept make use of the package validation systems that are used to confirm that the correct drug product has been packaged. The drug product characteristics that are obtained from the package validation process can be used to supplement the drug product information obtained from other database sources and may be stored together in the common drug and settings database 175. Information associated with the drug product is received from a drug product package validation system at block 705. In some embodiments, this information may comprise one or more images of the drug product and an identity of the drug product. The image may be generated, for example, at the drug product package validation system to validate a drug product package. The image of the drug product may be processed to acquire a plurality of characteristics associated with the drug product at block 710. The plurality of drug product characteristics may include, but are not limited to, size, shape, color, imprint code, weight, and/or scoring. The weight information may be obtained directly or indirectly through computations performed on other characteristics, such as size, drug product composition, and the like. The characteristics acquired from processing the image of the drug product are then associated with the identities of the different drug products in the common drug and settings database 175 at block 715. That is, the external database information for the various drug products is supplemented with the characteristics of these drug products and stored in an accessible data structure, such as a database. In some embodiments, the data structure or database may be a relational database, such as a Structured Query Language database. Thus, the common drug and settings database 175 may, in some embodiments, be configured to store information therein and provide selected information in response to queries applied thereto. In some embodiments, the common drug and settings database 175 may not be updated to include the characteristics of a particular drug product until these characteristics have been obtained from one or more drug product packaging validation systems a number of times that meets or exceeds a threshold.

In other embodiments, the drug product package validation system may acquire the plurality of characteristics associated with the drug product based on the one or more images as part of the drug product package validation process and communicate these characteristics in place of or in addition to one or more images for supplementing the common drug and settings database 175.

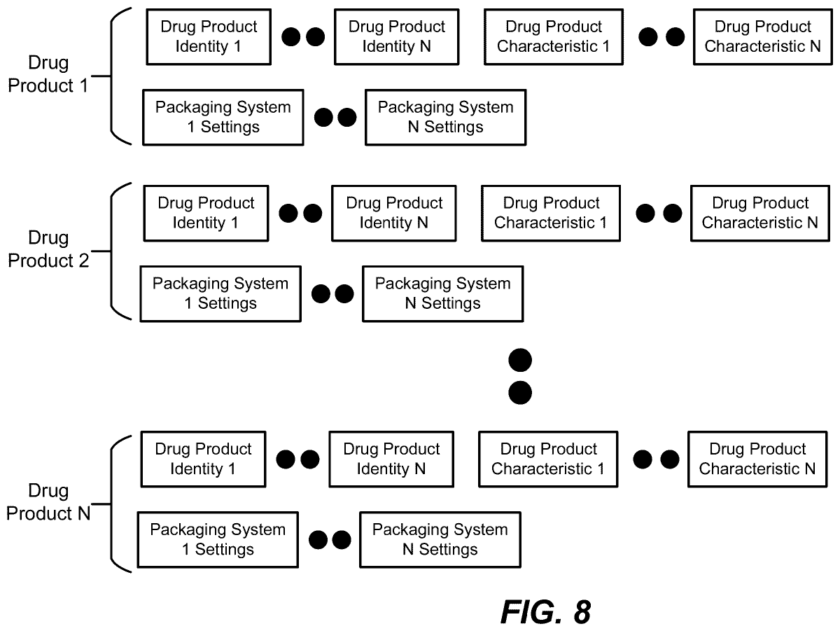

FIG. 8 is a block diagram of a common drug and settings database including heterogeneously obtained drug product information in accordance with some embodiments of the inventive concept. As shown in the example of FIG. 8, the drug product information, which is heterogeneously obtained from both drug product information repositories 170 and the drug product validation process performed using one or more drug product packaging systems 130a, 130b, includes individual records for each drug product. Each record includes multiple fields for each drug product, including one or more drug product identity fields, one or more drug product characteristics fields, and one or more packaging system settings fields. Multiple identity fields may be used for each drug product as a drug product may be identified using an NDC identifier, a DIN identifier, UPC identifier, or other type of identifier. Advantageously, the common drug and settings database 175 may be used to translate between identifiers used for the same drug product in different applications, countries, jurisdictions, and the like. The drug product characteristic fields may be used to hold the various drug product characteristics that are associated with a drug product. As the characteristics of a drug product may be different in different jurisdictions due to, for example, different inactive ingredients used in the drug products, different sets of characteristics, e.g., shape, size, color, and the like may be associated with the different identifiers for the different jurisdictions. As described above, in some embodiments, the drug product characteristics may comprise size, shape, color, imprint code, weight, and/or scoring. The imprint code characteristic may comprise an indicia or medicinal strength, an indicium of an active ingredient, and/or an indicium of an inactive ingredient. The shape characteristic may be round, elliptical, capsule, triangular, oblong, oval, square, diamond, hexagon, octagon, pentagon, gelcap, non-uniform, and/or other. The color characteristic may be any of a plurality of colors and/or transparent. Each record may include a field with settings that can be used to dispense the drug product on a particular drug product packaging system. These settings may include, but are not limited to, canister size and/or type, channel dimensions for the drug product, a baffle arrangement for the drug product, and/or a dispense channel air pressure for the drug product. As different types of drug product packaging systems may operate differently or use different settings, each record may include up to N different packaging system settings to support up to N different drug product packaging systems. Similarly, each record may include a field with a layout configuration that specifies the cell locations in a particular drug product packaging system for the drug product. As different types of drug product packaging systems are structured differently, each record may include up to N different packaging system configurations to support up to N different drug product packaging systems.

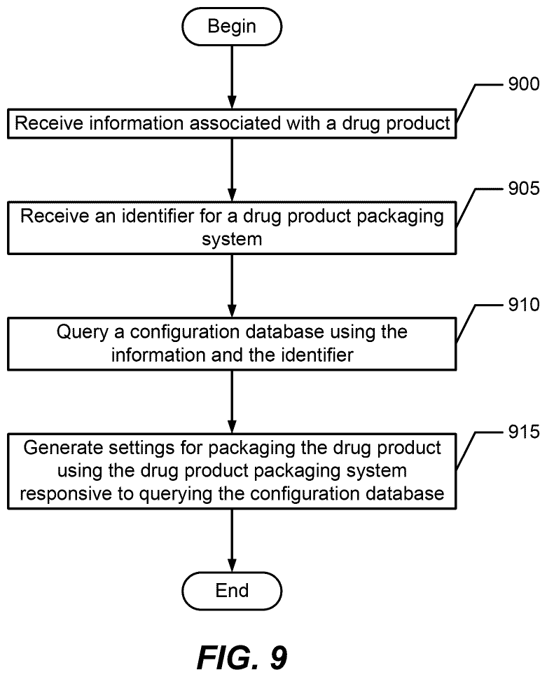

FIG. 9 is a flow chart that illustrates operations for generating dispense settings for a drug product packaging system based on drug product characteristics in accordance with some embodiments of the inventive concept. Referring now to FIG. 9, operations begin at block 900 where information is received that is associated with a drug product. In some embodiments, this information may include a drug product identifier along with details on one or more characteristics associated with the drug product. In other embodiments, the drug product may be scanned, for example, and the image processed as described above using an AI model to identify one or more characteristics of the drug product. An identifier for a drug product packaging system is received at block 905, which may be representative of a particular model or type of drug product packaging system. The information received or obtained, such as the drug product identifier and/or characteristics along with the identifier for the drug product packaging system are used to query a configuration database, such as the common drug and settings database 175 of FIG. 1 and FIG. 8, at block 910. In response to the query, dispense settings for packaging the drug product using the drug product packaging system corresponding to the identifier are generated at block 915. These settings may include one or more of canister size and/or type, dispense channel dimensions for the drug product, a baffle arrangement for the drug product, and/or a dispense channel air pressure for the drug product. In some embodiments, these settings can be provided to a technician who can make manual adjustments to apply the generated settings to the drug product packaging system. In other embodiments, a calibration system, either integrated in the drug product packaging system or stand alone, may be capable of adjusting the dispense settings for a drug product automatically. Thus, the dispense settings for packaging the drug product can be communicated to the automatic calibration system and application of the settings can be initiated on the drug product packaging system components responsive to their communication.

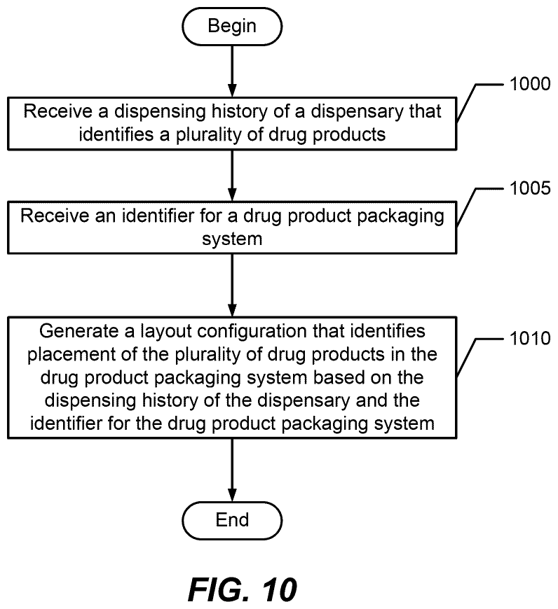

FIG. 10 is a flowchart that illustrates operations for generating a cell layout configuration for a drug product packaging system based on drug product characteristics in accordance with some embodiments of the inventive concept. As drug product packaging systems may include different types of cells for dispensing different types of drug products, the generated layout configuration may take into account the cell design within the drug product packaging system ensuring that a particular cell that is assigned to hold a particular type of drug product is capable of holding and dispensing the assigned drug product. Referring now to FIG. 10, operations begin at block 1000 where a dispensing history of a dispensary is received that identifies a plurality of drug products. The dispensing history may, in some embodiments, include a list of the drug products that have been dispensed through the dispensary. The dispensing history may further include the quantities of the various drug products that were dispensed along with the dates and/or times when the drug products were dispensed. In this way, the dispensing frequency may be derived from the dispensing history information. The dispensing history may be a list of drug products that have been dispensed manually by the dispensary. This may allow a new drug product packaging system to be configured for dispensing drug products that have previously been manually dispensed. In other embodiments, the dispensing history may identify drug products that have been dispensed by one or more drug product packaging systems. In still further embodiments, the dispensing history may identify a combination of drug products that have been dispensed manually and through a drug product packaging system. An identifier for a particular drug product packaging system is received at block 1005, which may be representative of a particular model or type of drug product packaging system. A layout configuration is generated at block 1010 that identifies placement of drug products in the drug product packaging system based on the dispensing history of the dispensary and the identifier for the drug product packaging system. Embodiments of generating the layout configuration for a drug product packaging system may be illustrated by way of example. A pharmacy, medical facility, hospital, or the like that operates a drug product packaging system may provide a dispensing history for a plurality of products dispensed and packaged using the drug product packaging system. For example, this may be a 90-day history of the prescriptions filled at a dispensary, such as a pharmacy. These may be filled manually and/or may be filed using the drug product packaging system or a similar drug product packaging system. The layout configuration may then be generated based on the drug products dispensed from the dispensary and identified in the dispensing history. The dispensing history may provide, for example, an indication of which drug products have higher dispense rates and which drug products have lower dispense rates. In some embodiments, the layout configuration may be generated such that the drug products that have higher dispense rates are placed in cell locations that operate as fast or faster than other ones of the cell locations in the drug product packaging system. In addition to generating a layout configuration that improves the speed of dispensing drug product, the generated layout configuration may also be designed to improve maintenance of the drug product packaging system. For example, cell locations that contain the same drug product may be placed adjacent to one another to allow the canisters or containers to be re-filled, for example, with fewer steps as the canisters or containers are not spread about the drug product packaging system. In some embodiments, canisters dedicated to the same drug product may all be located in a common drawer so that only a single drawer needs to be accessed to perform maintenance, e.g., a refill, on the canisters or containers associated with that drug product. The operations of FIG. 10 in generating a layout configuration may be used, for example, in setting up a new drug product packaging system for the first time or when a new drug product packaging system is replacing an old drug product packaging system. The operations of FIG. 10 in generating a layout configuration may also be used, for example, when a drug product packaging system is being re-configured to dispense a different selection of drug products, for example, at seasonal transition times, e.g., winter to spring, spring to summer, summer to fall, and/or fall to winter.

In some embodiments, a configuration database, such as the common drug and settings database 175 of FIG. 1 may be queried to obtain characteristic information for a drug product. The drug product size information may be obtained from this characteristic information and used to determine a canister size and/or type for storing the drug product in the drug product packaging system. In addition to the drug product size information, package quantity information may be used to determine the canister size. For example, if a drug product is typically packaged into a vial or bottle containing a particular quantity of drug product, then a canister may be chosen that is capable of holding enough drug product to fill a particular number of vials or bottles of the drug product. This determination may be made taking into account the number of pills or tablets held in the stock bottle typically purchased by the dispensary, as may be indicated in the dispensing history.

While the operations of FIG. 10 in generating a layout configuration for a drug product packaging system to dispense or package a plurality of drug products, the operations can also be applied at a higher level with respect to assigning drug products to particular packaging system. For example, a hospital may have multiple drug product packaging systems located throughout a facility. Drug products associated with heart disease may be located in a drug product packaging system that is closer to the cardiology department. Similarly, drug products associated with allergy care may be located in a drug product packaging system that is closer to an allergy and immunology department.

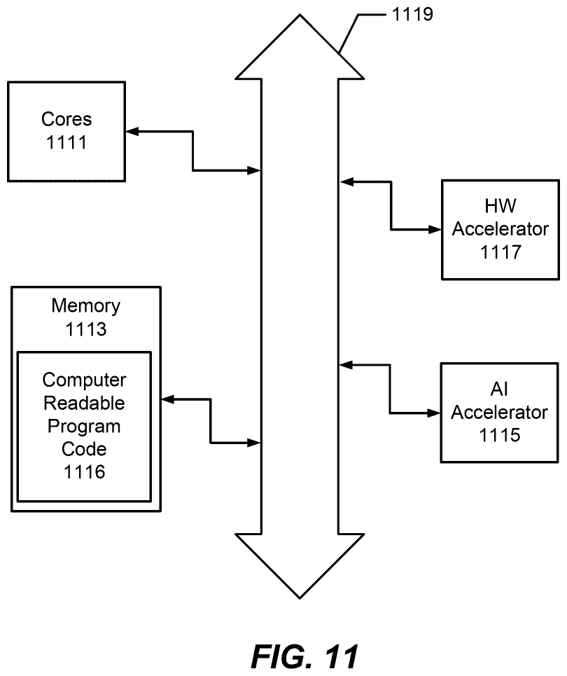

FIG. 11 is a block diagram of a data processing system that may be used to implement the drug product analysis and packaging system configuration server 155 of FIG. 1 in accordance with some embodiments of the inventive concept. As shown in FIG. 11, the data processing system may include at least one core 1111, a memory 1113, an artificial intelligence (AI) accelerator 1115 and a hardware (HW) accelerator 1117. The at least one core 1111, the memory 1113, the AI accelerator 1115, and the HW accelerator 1117 may communicate with each other through a bus 1119.

The at least one core 1111 may be configured to execute computer program instructions. For example, the at least one core 1111 may execute an operating system and/or applications represented by the computer readable program code 1116 stored in the memory 1113. In some embodiments, the at least one core 1111 may be configured to instruct the AI accelerator 1115 and/or the HW accelerator 1117 to perform operations by executing the instructions and obtain results of the operations from the AI accelerator 1115 and/or the HW accelerator 1117. In some embodiments, the at least one core 1111 may be an ASIP customized for specific purposes and support a dedicated instruction set.

The memory 1113 may have an arbitrary structure configured to store data. For example, the memory 1113 may include a volatile memory device, such as dynamic random-access memory (DRAM) and static RAM (SRAM), or include a non-volatile memory device, such as flash memory and resistive RAM (RRAM). The at least one core 1111, the AI accelerator 1115, and the HW accelerator 1117 may store data in the memory 1113 or read data from the memory 1113 through the bus 1119.

The AI accelerator 815 may refer to hardware designed for AI applications. In some embodiments, the AI accelerator 1115 may include one or more AI models configured to facilitate validation of a packaged and/or unpackaged drug product, collect drug product identifiers and associated drug product characteristics from the validation process, compile the collected information along with drug product identities and characteristics obtained from drug product information repositories 170 into a common drug and settings database 175, and generate drug product dispense settings and/or drug product layout configurations for one or more drug product packaging systems. The AI accelerator 1115 may generate output data by processing input data provided from the at least one core 1115 and/or the HW accelerator 1117 and provide the output data to the at least one core 1111 and/or the HW accelerator 1117. In some embodiments, the AI accelerator 1115 may be programmable and be programmed by the at least one core 1111 and/or the HW accelerator 1117. The HW accelerator 1117 may include hardware designed to perform specific operations at high speed. The HW accelerator 1117 may be programmable and be programmed by the at least one core 1111.

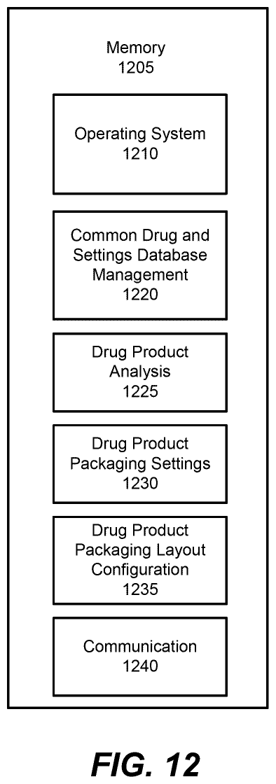

FIG. 12 illustrates a memory 1205 that may be used in embodiments of data processing systems, such as the drug product analysis and packaging system configuration server 155 of FIG. 1 and the data processing system of FIG. 11, respectively, to facilitate validation of a packaged and/or unpackaged drug product, collect drug product identifiers and associated drug product characteristics from the validation process, compile the collected information along with drug product identities and characteristics obtained from drug product information repositories 170 into a common drug and settings database 175, and generate drug product dispense settings and/or drug product layout configurations for one or more drug product packaging systems according to some embodiments of the inventive concept. The memory 1205 is representative of the one or more memory devices containing the software and data used for facilitating operations of the drug product analysis and packaging system configuration server 155 and the drug product analysis and packaging system configuration module 160 as described herein. The memory 1205 may include, but is not limited to, the following types of devices: cache, ROM, PROM, EPROM, EEPROM, flash, SRAM, and DRAM. As shown in FIG. 12, the memory 1205 may contain six or more categories of software and/or data: an operating system 1210, a common drug and settings database management module 1220, a drug product analysis module 1225, a drug product packaging settings module 1230, a drug product packaging layout configuration module 1235, and a communication module 1240. In particular, the operating system 1210 may manage the data processing system's software and/or hardware resources and may coordinate execution of programs by the processor. The common drug and settings database management module 1220 may be configured to manage the storage of information in the common drug and settings database 175 and to query the common drug and settings database 175 to obtain information therefrom. Accordingly, the common drug and settings database management module 1220 may be configured to perform one or more of the operations described above with respect to the common drug and settings database 175 of FIGS. 1 and 8 and the flowcharts of FIGS. 7, 9, and 10. The drug product analysis module 1225 may be configured to perform one or more of the operations described above with respect to the machine learning engine 240, the convolutional neural network 310, and the flowcharts of FIGS. 7 and 9. The drug product packaging setting module 1230 may be configured to perform one or more of the operations described above with respect to the common drug and settings database 175 of FIGS. 1 and 8 and the flowchart of FIG. 9. The drug product packaging layout configuration module 1235 may be configured to perform one or more of the operations described above with respect to the common drug and settings database 175 of FIGS. 1 and 8 and the flowchart of FIG. 10.

Although FIGS. 11-12 illustrate hardware/software architectures that may be used in data processing systems, such as the drug product analysis and packaging system configuration server 155 of FIG. 1 and the data processing system of FIG. 11, respectively, in accordance with some embodiments of the inventive concept, it will be understood that embodiments of the inventive concept are not limited to such a configuration but are intended to encompass any configuration capable of carrying out operations described herein.

Computer program code for carrying out operations of data processing systems discussed above with respect to FIGS. 1-12 may be written in a high-level programming language, such as Python, Java, C, and/or C++, for development convenience. In addition, computer program code for carrying out operations of embodiments of the inventive concept may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the drug product analysis and packaging system configuration server 155 of FIG. 1 and the data processing system of FIG. 11 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-12 may be used to facilitate v validation of a packaged and/or unpackaged drug product, collect drug product identifiers and associated drug product characteristics from the validation process, compile the collected information along with drug product identities and characteristics obtained from drug product information repositories 170 into a common drug and settings database 175, and generate drug product dispense settings and/or drug product layout configurations for one or more drug product packaging systems according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1205 when coupled to a processor includes computer readable program code that, when executed by the processor, causes the processor to perform operations including one or more of the operations described herein with respect to FIGS. 1-10.

As described above, some embodiments of the inventive concept may provide a data structure, such as a database containing drug product identifiers along with their associated characteristics, drug product packaging system dispensing setting information, for each of the drug products, that may be configured in a computer readable storage medium where it can be accessed by query. The data structure or database can be populated with information from drug product information repositories and supplemented with drug product characteristic information obtained, for example, when validating drug products during the packaging process. Thus, the data structure or database can be effectively populated by collecting drug product identities from one or more commercially available and/or proprietary databases and supplementing this information with drug product characteristics that are collected across one or more drug product packaging systems. The data structure or database may be used as a resource for managing or configuring the settings for dispensing the various drug products. Moreover, in other embodiments, a dispensing history of a dispensary that identifies a plurality of drug products may be obtained and used for configuring the layout or placement of drug products in cells in the drug product packaging system to improve the operating efficiency in both dispensing drug product and the maintenance of the drug product packaging system, e.g., managing re-fills of drug product containers.

### Further Definitions and Embodiments

In the above-description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods, and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the disclosure. As used herein, the singular forms “a”, “an” and “the” are intended to include the plural forms as well, unless the context clearly indicates otherwise. It will be further understood that the terms “comprises,” “comprising,” “include”, “including”, “includes”, “have”, “has”, “having”, or variants thereof when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

It will also be understood that, although the terms first, second, etc. may be used herein to describe various elements, these elements should not be limited by these terms. These terms are only used to distinguish one element from another.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

1. A method comprising:
receiving information associated with a drug product;
receiving an identifier for a drug product packaging system;
querying a configuration database using the information and the identifier; and
generating settings for packaging the drug product using the drug product packaging system responsive to querying the configuration database.

2. The method of claim 1, wherein the information comprises an identity of the drug product.

3. The method of claim 2, wherein querying the configuration database comprises querying the configuration database using the identity of the drug product and the identifier.

4. The method of claim 2, wherein each of the plurality of characteristics comprises size, shape, color, imprint code, or scoring.

5. The method of claim 4, wherein the imprint code comprises an indicium of medicinal strength, an indicium of an active ingredient, and an indicium of an inactive ingredient.

6. The method of claim 4, wherein the shape comprises round, elliptical, capsule, triangular, oblong, oval, square, diamond, hexagon, octagon, pentagon, gelcap, non-uniform, and other.

7. The method of claim 4, wherein the color comprises transparent and a plurality of colors.

8. The method of claim 2, wherein the identity comprises a National Drug Code (NDC), a Drug Identification Number (DIN), or a Universal Product Code (UPC) identifier.

9. The method of claim 1, wherein the settings comprise one or more of dispense channel dimensions for the drug product, a baffle arrangement for the drug product, or a dispense channel air pressure for the drug product.

10. The method of claim 1, further comprising: communicating the settings to the drug product packaging system; and initiating application of the settings on the drug product packaging system responsive to communicating the settings to the drug product packaging system.

11. A method comprising:
receiving a dispensing history of a dispensary that identifies a plurality of drug products;
receiving an identifier for a drug product packaging system; and
generating a layout configuration that identifies placement of the plurality of drug products in cell locations in the drug product packaging system based on the dispensing history of the dispensary and the identifier for the drug product packaging system.

12. The method of claim 11, further comprising:
querying a configuration database using an identity of one of the plurality of drug products to obtain size information for the one of the plurality of drug products; and
determining a cannister size for the one of the plurality of drug products based on the size information;
wherein generating the layout configuration comprises:
generating the layout configuration based on the dispensing history and the cannister size for the one of the plurality of drug products.

13. The method of claim 12, wherein determining the cannister size comprises:
determining the cannister size for the one of the plurality of drug products based on the size information and package quantity information for the one of the plurality of drug products.

14. The method of claim 11, wherein generating the layout configuration comprises:
generating the layout configuration with ones of the plurality of drug products having higher dispense rates at ones of the cell locations that operate as fast or faster than other ones of the cell locations.

15. The method of claim 11, wherein generating the layout configuration comprises:
generating the layout configuration such that ones of the cell locations that contain a same one of the plurality of drug products are adjacent to one another.

16. The method of claim 15, wherein generating the layout configuration such that the ones of the cell locations that contain the same one of the plurality of drug products are adjacent to one another comprises:
generating the layout configuration such that the ones of the cell locations that contain the same one of the plurality of drug products are in a common drawer.

17. The method of claim 11, wherein the identity comprises a National Drug Code (NDC), a Drug Identification Number (DIN), or a Universal Product Code (UPC) identifier.

18. A system, comprising:
a processor; and
a memory coupled to the processor and comprising computer readable program code embodied in the memory that is executable by the processor to perform operations comprising:
receiving information associated with a drug product;
receiving an identifier for a drug product packaging system;
querying a configuration database using the information and the identifier; and
generating settings for packaging the drug product using the drug product packaging system responsive to querying the configuration database.

19-21. (canceled)
